(19) United States
(12) Patent Application Publication
(10) Pub. No.: US 2007/0116745 A1
(43) Pub. Date: May 24, 2007

(54) Spermicidal preparations and uses thereof

(75) Inventor: John Dawson, East Fremantle (AU)

Correspondence Address: MARSHALL, GERSTEIN & BORUN LLP, 233 S. WACKER DRIVE, SUITE 6300, SEARS TOWER, CHICAGO, IL, 60606, US

(73) Assignee: CONVE LTD., Perth (AU)

(21) Appl. No.: 10/590,070

(22) PCT Filed: Feb. 16, 2005
(86) PCT No.: PCT/AU05/00197

§ 371 Date: Nov. 13, 2006

(30) Foreign Application Priority Data
Feb. 16, 2004 (AU) 20049007-19

(51) Int. Cl. A61K 33/34; A61K 9/70

(52) U.S. Cl. 424/443; 424/638

(57) ABSTRACT

A method of controlling sperm comprising the step of contacting the sperm with an effective amount of copper silicate.

### FIELD OF THE INVENTION

The present invention relates to the use of copper silicate compositions active against sperm and in particular compositions that are able to kill and/or retard sperm motility. The present invention also relates to contraceptive compositions comprising copper silicate adapted for topical administration. The present invention also relates to contraceptive devices impregnated or otherwise treated to contain copper silicate.

### BACKGROUND

Spermicides exert an anti-fertility effect upon spermatozoa as it passes through the female genital tract. To be an effective contraceptive agent, a compound must meet essential requirements. It must act rapidly and efficiently to kill or immobilize sperm on contact, or render sperm incapable of fertilization. It also should (i) be suitable for administration in terms of not being unduly irritating to the vaginal and penile mucosa (ii) not have any adverse effect on a developing embryo or fetus, and (iii) be free of long-term toxicity. Moreover, it should be systemically non-toxic.

At present, commercially available spermicidal contraceptives have detergent ingredients that disrupt cell membranes (due to their affinity to the membrane lipids). These include the neutral surfactants nonoxynol-9 (N-9), menfegol, and octoxynol-9 (O-9). N-9 is the most commonly used spermicidal contraceptive in the UK and the USA. The cationic surfactant benzalkonium chloride and the anionic detergent sodium docusate are also used worldwide as vaginal spermicides. Octoxynol-9 is currently the only neutral surfactant present on the Australian market. Its properties are presented as equivalent to that of N-9.

Unfortunately, these detergents and in particular N-9 have been shown to damage the cell lining of the vagina and cervix, thereby increasing the risk of STD transmission. In several studies conducted in African countries where HIV is endemic, the use of N-9 based spermicides has been linked to a greater risk of HIV transmission. In this regard, the USA Food and Drug Administration (FDA) has recently proposed a warning for all contraceptives containing N-9. This warning includes advice to consumers that the use of vaginal contraceptives containing N-9 can increase vaginal irritation, which may actually increase the possibility of transmitting the AIDS virus and other STDs from infected partners.

There exists a need for improved contraceptives which are sperm-active but do not suffer from one or more of the deficiencies of currently available products. The present invention seeks to overcome the above problems by providing safe and effective sperm-active compositions, including topical formulations that can be used to control sperm.

### SUMMARY OF THE INVENTION

The present invention provides a method of controlling sperm, the method comprising the step of contacting the sperm with an effective amount of copper silicate.

The ability of copper silicate to control sperm renders it useful in applications where it is desirous to reduce or totally remove sperm motility or otherwise inactivate sperm. One particular application where this activity is useful is in the production of contraceptives. Thus, the present invention also provides for the use of an effective amount of copper silicate as a contraceptive.

The copper silicate used in the methods of the present invention may be formulated to render them particularly suitable for administration to mammals such as humans. Thus, the present invention also provides for the use of copper silicate for the preparation of a formulation for use as a contraceptive and a composition adapted for topical administration comprising an effective amount of copper silicate wherein the effective amount is sufficient to act as a contraceptive.

The copper silicate used in the method of the present invention may also be combined or otherwise integrated into existing contraceptive devices such as barrier agents to improve their effectiveness as a contraceptive. Thus, the present invention also provides a contraceptive device comprising copper silicate.

### DETAILED DESCRIPTION OF THE INVENTION

Methods of Controlling Sperm

The present invention provides a method of controlling sperm, the method comprising the step of contacting the sperm with an effective amount of copper silicate.

For the purposes of the present invention, the phrase “controlling sperm” and similar phrases such as “controls sperm” means one or more of the following: at least reducing sperm motility, at least reducing the number of viable sperm, at least reducing the ability to penetrate cervical mucous and killing sperm.

The ability of copper silicate to control sperm renders it useful as a contraceptive. The present invention also provides for the use of an effective amount of copper silicate as a contraceptive.

When used as a contraceptive the copper silicate may be applied in a variety of ways so that it contacts and controls the sperm. For example the copper silicate may be applied to a site expected to receive or come into contact with sperm. Thus, the site may be a body part such as a reproductive organ or part thereof and in particular the site may be part of the reproductive tract, the penis, vagina or cervix. Alternatively, the site may be a physical object such as another contraceptive agent such as a condom, diaphragm or the like or a sex aid.

The effective amount of the copper silicate applied in the method of the present invention will vary depending, at least, on the application site and the conditions at that site. However, it will at least be sufficient to control sperm. Examples of the amount of copper silicate product applied according to the method of the present invention are weight range: 1-10 g, 2-8 g or 4-6 g.

The frequency with which, and the duration for which, the copper silicate is applied will be sufficient to control sperm and thus will also vary depending at least on the site of application and the concentration of the copper silicate. It is expected the copper silicate will be applied on a needs basis by the end user to meet specific requirements.

Formulations

To render them particularly suitable for application to mammals such as humans the copper silicate used in the methods of the present invention may be specially formulated. Thus, the present invention also provides for the use of copper silicate for the preparation of a formulation for controlling sperm or use as a contraceptive.

The formulations of the present invention may be produced by dissolving or combining the copper silicate in an aqueous or non-aqueous carrier. In general, any liquid, cream, or gel, or similar substance that does not appreciably react with the copper silicate or any other active ingredient that may be introduced and which is non-irritating is suitable.

The formulations may be adapted for administration via a range of routes. However, preferably, the formulations are adapted for topical administration. Thus, the present invention also provides, a method of producing a compound adapted for topical administration comprising the step of dissolving or combining copper silicate in an aqueous or non-aqueous topical carrier.

The present invention also provides a formulation adapted for topical administration comprising an effective amount of copper silicate. For the purposes of the present invention, the term “topical” means application to a localized area of the body and/or to the surface of a body part and includes administration to the vagina (such as intra-vaginally) and to the mucous membranes.

The form of the copper silicate in the formulation of the present invention may be varied provided it retains its ability to control sperm. Preferably, the copper silicate is present in the formulation as a solution. Acidified solutions, including aqueous solutions, are particularly preferred because copper silicate is more soluble and stable at acidic pH. Particularly preferred pHs are 3-6, 4-6 and 5-6. However, it will be appreciated that the pH of the formulation should be physiologically acceptable.

The formulations may be rendered acidic through the addition of acids that are therapeutically acceptable in terms of not unduly comprising the effectiveness of the formulation by being overly irritating or causing other undesirable side-effects. A particularly preferred therapeutically acceptable acid for the purposes of the present invention is acetic acid. The final concentration of acetic acid in the formulations of the present invention may be varied and preferably are between about 0.1% wt and 2% wt and more preferably 0.5% wt and 1.5% wt.

The copper silicate may also be in solid form provided it is properly prepared. In this regard, the copper silicate could be in the form of a micronized solid such as chrysocolla.

The composition adapted for topical administration may be in the form of any one of the following: solution, lotion, suspension, emulsion, cream, gel, ointment, liniment and salve. Particularly preferred forms are ointments, creams or gels.

Ointments generally are prepared using either (1) an oleaginous base, i.e., one consisting of fixed oils or hydrocarbons, such as white petroleum or mineral oil, or (2) an absorbent base, i.e., one consisting of an anhydrous substance or substances that can absorb water, for example anhydrous lanolin. Customarily, following formation of the base, whether oleaginous or absorbent, the active ingredient is added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface active agent, such as sodium lauryl sulfate; hydrophilic colloids, such as acacia colloidal clays, veegum and the like. For the purposes of the present invention, the compound may be added to the water phase prior to formation of the emulsion, in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. For the purposes of the present invention the compound may be added to the formulation at the desired concentration at a point preceding addition of the gelling agent. Preferably, the formulations of the present invention have lubricant characteristics. Thus, the present invention also provides a formulation adapted for topical administration comprising an effective amount of copper silicate wherein the formulation is adapted to also act as a lubricant. The formulations of the present invention will often have lubricant characteristics inherently due to other agents in the formulation. However, this aspect of the invention also covers lubricants that have copper silicate incorporated therein.

The formulations of the present invention may further comprise an auxiliary agent such as any one or more of: preservatives, stabilizers, emulsifiers, wetting agents, fragrances, colouring agents, odour controllers and thickeners such as natural gums.

The concentration of the copper silicate in the formulation may be varied as required and with reference to the intended end use. However, preferably, the concentration of the copper silicate is such that its final concentration is approximately 0.01% -10% w/w (as Cu). More preferably, the concentration of the copper silicate is to a final concentration of approximately 0.05% -0.5% w/w (as Cu) or 0.05% -0.3% (as Cu).

The formulations of the present invention include those that are adapted for delivery via a solid dosage form such as a tablet or suppository. Thus, the present invention also provides a solid dosage form such as a tablet or suppository or the like comprising copper silicate or a formulation thereof.

Solid dosage forms suitable for the purposes of the present invention are described generally in Martin, Remington's Pharmaceutical Sciences, 18th Ed. (1990 Mack Publishing Co. Easton Pa. 18042) which is herein incorporated by reference. These include tablets, capsules and pellets.

Disintegrants may be included in the solid dosage form. Materials used as disintegrants include but are not limited to starch including the commercial disintegrant based on starch, Explotab. Sodium starch glycolate, Ambedite, sodium carboxymethylcellulose, ultramylopectin, sodium alginate, gelatine, orange peel, acid carboxymethyl cellulose, natural sponge and bentonite may all be used. Another form of the disintegrants are insoluble cationic exchange resins. Powdered gums may be used as disintegrants and as binders and these can include powdered gums such as agar, Karaya or tragacanth. Alginic acid and its sodium salt are also useful as disintegrants.

An antifrictional agent may be included in the formulation to prevent sticking during the formulation process. Lubricants may be used as a layer between the copper silicate and the die wall and these can include but are not limited to: stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights and Carbowax 4000 and 6000.

Glidants that might improve the flow properties of the composition during formulation and to aid rearrangement during compression might be added. The glidants may include starch, talc, pyrogenic silica and hydrated silicoaluminate.

To aid dissolution of the copper silicate into the aqueous environment, a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compositions either alone or as a mixture in different ratios.

Controlled release formulations may be desirable. The compositions could be incorporated into an inert matrix that permits release by either diffusion or leaching mechanisms such as gums. Slowly degenerating matrices may also be incorporated into the formulation. Another form of a controlled release is by a method where the copper silicate is enclosed in a semipermeable membrane that allows water to enter and push the copper silicate out through a single small opening due to osmotic effects. Some enteric coatings also have a delayed release effect.

A mix of materials might be used to provide the optimum film coating. Film coating may be carried out in a pan coater or in a fluidised bed or by compression coating.

The copper silicate can also be included in the formulation as multiparticulates such as granules or pellets of particle size about 1 mm. Thus, the invention further provides for formulations comprising microparticles, created from hydrophilic polymers, which contain copper silicate. The microparticles containing the copper silicate may be made by a variety of methods known to those in the art, for example, solvent evaporation, desolvation, complex coacervation, polymer/polymer incompatibility and interfacial polymerisation.

Devices

The copper silicate may also be incorporated into or applied to other contraceptive devices such as barrier agents to improve their contraceptive capacity. Thus, the present invention also provides a contraceptive device comprising copper silicate.

The devices of the present invention may be varied provided they are adapted to receive or be treated in a fashion that enables them to incorporate copper silicate and later make the copper or copper silicate bioavailable in a manner that enables it to control sperm. Preferably, the device is a barrier agent such as an agent selected from the group consisting of: sponges, films, cervical caps, diaphragms and condoms.

When the copper silicate is incorporated into a device it may be incorporated into the matrix of the material from which the device is made or it may be applied as a coating on the device. This may be relatively simple in the case of a sponge. However, when the device is a condom incorporating the copper silicate into the rubber matrix may involve some trial and error to ensure the copper is bioavailable. Regardless, armed with the information herein a person skilled in the art can produce the devices of the present invention through routine trial and experiment.

The present invention will now be described with reference to the following examples. The description of the examples is in no way to limit the generality of the preceding description.

### EXAMPLES

### Example 1

Spermicidal Activity of Copper Silicate Formulations

Materials/Methods

The following products according to the invention were used in the examples.

| | | Approx. Cu content |
|---|---|---|
| Identifier | Description/Form | (as Cu % w/w) |
| CSG5 | Gel formulation | 0.188 |
| CSG4 | Gel formulation | 0.094 |
| CSL1 | Lotion | 0.24 |
| CSSOL1 | Solution | 0.28 |

All these products contain copper in the form of soluble copper silicate. The formulations all use a concentrated solution of copper silicate as the source of the active copper silicate.

CSC - Concentrated Copper Silicate Solution

| Ingredient | % wt |
|---|---|
| Deionised Water | 87.45 |
| Copper sulfate pentahydrate | 4.35 |
| Acetic acid (90%) | 3.60 |
| Sodium silicate solution | 4.60 |

CSSOL1

| Ingredient | % wt |
|---|---|
| CSC | 25.22 |
| Water | 74.73 |
| Sodium alkyl ether sulfate | 0.05 |

CSG4

| Ingredient | % wt |
|---|---|
| Deionised Water | 66.88 |
| CSC | 7.89 |
| Triethanolamine | 1.31 |
| Carbopol Ultrez 10 | 3.55 |
| Glycerine | 10.45 |
| Propylene Glycol | 9.40 |
| Germaben IIE | 0.52 |

CSG5

| Ingredient | % wt |
|---|---|
| Deionised Water | 58.52 |
| CSC | 14.94 |
| Triethanolamine | 2.62 |
| Carbopol Ultrez 10 | 3.55 |
| Glycerine | 10.45 |
| Propylene Glycol | 9.40 |
| Germaben IIE | 0.52 |

CSL1

| Ingredient | % wt |
|---|---|
| Deionised Water | 56.72 |
| CSC | 19.95 |
| Triethanolamine | 1.10 |
| Carbopol Ultrez 10 | 1.89 |
| Glycerine | 9.97 |
| Alcohol | 6.98 |
| Sodium hydroxide | 3.39 |
| solution 18% | |

The above formulations were used in a series of (i) Sander-Cramer assays (assessment of complete sperm immobilization during a 30-second, compound-sperm coincubation); and (ii) cervical mucous (CM) penetration assays (compound is pre-incubated with a CM microcolumn for 30 minutes, after which sperm are introduced into the system; sperm migration through the mucous is compared to that of control).

Results

The results are shown in Tables 1 and 2 hereunder.

TABLE 1

| | | MODIFIED SANDER-CRAMER ASSAY | | | | |
|---|---|---|---|---|---|---|
| | | | HIGHEST | M.E.C. | | |
| | | INITIAL CONC | SPERMICIDAL | (% w/w as | | |
| COMPOUND | SOLVENT | (% w/w as Cu) | DILUTION (1/X) | Cu) | n | SOLUBILITY |
| CSG4 | 0.9% NaCl* | 0.09 | 3.7 ± 0.3 | 0.02 | 6 | Blue-green gel |
| CSG5 | 0.9% NaCl* | 0.16 | 2.7 ± 0.4 | 0.06 | 6 | Blue-green gel |
| CSSOL1 | 0.9% NaCl* | 0.28 | 8.0 ± 0.0 | 0.04 | 6 | Blue solution |
| CSL1 | 0.9% NaCl* | 0.22 | 8.0 ± 0.0 | 0.03 | 6 | Blue-green |
| | | | | | | lotion |
| | | | HIGHEST | | | |
| | | | SPERMICIDAL | | | |
| | | INITIAL CONC | DILUTION | M.E.C. | | |
| COMPOUND | SOLVENT | (mg/ml) | (1/X) | (mg/ml) | n | SOLUBILITY |
| Nonoxynol-9 | 0.9% NaCl* | 1 | 8.0 ± 0.0 | 0.125 ± 0.000 | 6 | OK - clear |
| | | | | | | solution |

M.E.C. = Minimum Effective Concentration
*Saline was pH to 5 with 0.1 N HCl. This saline was used for serial dilutions also.

TABLE 2

| | |
|---|---|
| Concentration | MOET |

| Compound | (dilution) | % CTL | n |
|---|---|---|---|
| CGS4 | 1:16 | 1.5 ± 1.0 | 10 |
| CGS5 | 1:16 | 13.9 ± 6.0 | 10 |
| CSL1 | 1:16 | 54.3 ± 8.7 | 10 |
| CSSOL1 | 1:16 | 17.7 ± 3.6 | 10 |
| 0.9% NaCl | | 100.0 ± 0.0 | 10 |

MOET: Modified One-End Test
% CTL: percent penetration of test sperm in cervical mucous as compared to that of solvent (0.9% NaCl) control spermatozoa.
Incubation volumes: 100 ul
Soln: 100 ul Adjusted Semen (60 mill/mL)
Values represent Mean ± Standard Error.

### Example 2

Spermicidal Formulations

Materials/Methods

1. Sperm Specimens

Raw sperm was washed and re-suspended in sterile culture medium. Three different donors were used throughout the study.

2. Sperm Testing

(a) Vitality Assessment

Every sperm sample underwent a vitality assessment consisting of a microscopic examination (normal light) of the cells, with the recording of their motility (qualitative assessment) and the percentage of motile cells (quantitative assessment). The assessment of the effect of the spermicides tested (copper silicate or “reference” spermicide—Octoxinol-9) was conducted against the untreated sperm. When assessing the effect of the spermicide formulations, only an approximation of the quality of the motility of the cells was given, as the immobilization of the sperm was the only critical parameter to be monitored in all tests.

(b) Spermicide Dilutions Testing

All spermicide formulations tested were serial diluted (v/v) with sterile 0.9% NaCI. Each dilution was mixed v/v with processed sperm (100 μL/100 μL or 50 μL/50 μL, depending on the original sperm concentration and volume). The first dilution (1/1) corresponds to the mixing of one volume of sperm suspension with one volume of pure CSSOL1 or 1 mg/mL octoxynol-9. Dilution 1/2 corresponds to the mixing of one volume of sperm suspension with one volume of half-strength CSSOL1or half-strength 1 mg/mL octoxynol-9, and so on.

An average of 100 cells was counted to obtain meaningful information upon sperm motility. Sperm was examined at 1 minute or 5 and 15 minutes (see results) after mixing with the spermicide dilution. Viability and resuscitation tests were carried out in some experiments. These tests helped to understand and relate reversible and irreversible sperm immobilization.

(i) Viability Testing

This test consisted of mixing sperm suspensions with two dyes (Eosin and Negrosin). The brightirefringent cells were ticked as viable, all the others (partial or total brownish coloration) as dead. This test was conducted on visually immobilized sperm.

(ii) Resuscitation Testing

Treated sperms were re-suspended in culture medium (1 v/20 v) for 15 minutes, then pelleted down by centrifugation (2000 RPM-200-300 g). The supernatant was carefully removed (pipette) and the pellet re-suspended in ˜100 μL of remaining liquid. The percentage of motile cells was then recorded as described above.

(c) Formulations Used

CSSOL1

| Ingredient | % wt |
|---|---|
| Water | 96.87 |
| Copper sulfate pentahydrate | 1.10 |
| Acetic acid | 0.82 |
| Sodium silicate solution | 1.16 |
| Sodium alkyl ether sulfate | 0.05 |

Solution B3:

| Ingredient | % wt |
|---|---|
| Water | 96.40 |
| Copper sulfate pentahydrate | 1.10 |
| Acetic acid | 1.34 |
| Sodium silicate solution | 1.16 |

LACSSOL

| Ingredient | % wt |
|---|---|
| Water | 96.51 |
| Copper sulfate pentahydrate | 1.10 |
| Lactic acid | 1.23 |
| Sodium silicate solution | 1.16 |

LACSSOL 150%

| Ingredient | % wt |
|---|---|
| Water | 95.89 |
| Copper sulfate pentahydrate | 1.10 |
| Lactic acid | 1.85 |
| Sodium silicate solution | 1.16 |

Note that in these tests all copper silicate based formulations were adjusted to pH 4 using 1 N NaOH prior to testing.

Ortho-gynol®

Ortho-gynol® contains Octoxinol-9 at a concentration of 10 mg/g. The dilutions tested were made in 0.9% NaCl from the 1 mg/mL working solution.

Results

Abbreviations

M: Motility—percentage indicates the proportion of motile cells, +and—indicate the average quality of this motility, ranging from ±(low, inefficient motility. The cells move/spin on themselves but not forward) to ++++(very quick forward movement). Cells tagged±are considered incapable of fertilization.

V: Viability—percentage indicates the proportion of viable cells as defined above.

Res: Resuscitation—describes the motility of the cells following the resuscitation procedure described above. The percentage indicates the proportion of motile cells. The average quality of this motility is ranked from ±to ++++, as described above.

nd: Not done.

The first effective spermicidal dilution is that which achieves 0% motility or inefficient motility (±) of sperm.

TABLE 3
Comparative spermicidal activity of CSSOL1-pH 4 and Ortho-gynol
(Donor A)

| | | | Ortho-gynol ® [Octoxinol-9] | |
|---|---|---|---|---|
| | CSSOL1 pH 4 | | (1 mg/mL) | |
| Dilution | 5 min | 15 min | 5 min | 15 min |
| 1/1 | M: 0% | M: nd | M: 0% | M: nd |
| | V: ˜0% | V: 0% | V: ˜0% | V: 0% |
| 1/2 | M: 0% | M: nd | M: 0% | M: nd |
| | V: 0% | V: nd | V: 0% | V: nd |
| 1/4 | M: 0% | M: 0% | M: ˜50%, ± | M: 0% |
| | V: 80% | V: 65% | V: ˜50% | V: ˜0% |
| 1/8 | M: 50%, ± | M: 35%, ± | M: >80%, +++ | M: >50%, ++ |
| | V: nd | V: 80% | V: nd | V: nd |
| 1/16 | M: >80%, +++ | M: >80%, ++ | M: | M: |
| | V: nd | V: nd | V: | V: |

TABLE 4
Second experiment on the comparative spermicidal activity of CSSOL1-
pH 4 and Ortho-gynol (Donor A)

| | |
|---|---|
| CSSOL1 pH 4 | Ortho-gynol ® [Octoxinol-9] (1 mg/mL) |

| Dilution | 5 min | 15 min | 5 min | 15 min |
|---|---|---|---|---|
| 1/1 | M: 0% | M: nd | M: 0% | M: nd |
| | V: 0% | V: nd | V: 0% | V: nd |
| 1/2 | M: 0% | M: nd | M: 0% | M: nd |
| | V: ˜20% | V: 0-20% | V: 0% | V: nd |
| | Res: 0% | Res: nd | Res: nd | Res: nd |
| 1/4 | M: 0% | M: 0% | M: 0% | M: nd |
| | V: 40% | V: 20-40% | V: ˜0% | V: nd |
| | Res: >50%, ++ | Res: ˜20-30%, ++ | Res: ˜0% | Res: nd |
| 1/8 | M: ≧50%, + | M: 50%, + | M: >50%, ++ | M: >50%, ++ |
| | V: nd | V: 80% | V: nd | V: nd |
| | Res: nd | Res: nd | Res: nd | Res: nd |

TABLE 5
Comparison of spermicidal activities of 3 copper silicate solutions
(Donor A)

| | CSSOL1 pH 4 | Solution B | Solution B3 |
|---|---|---|---|
| Dilution | 1 min | 1 min | min |
| 1/1 | M: 0% | M: 0% | M: 0% |
| | Res: nd | Res: nd | Res: nd |
| 1/2 | M: 0% | M: 0% | M: 0% |
| | Res: 20-30%, ±/+ | Res: ≦20%, ± | Res: ≦20%, ± |
| 1/4 | M: ≦10%, ± | M: 0% | M: 0% |
| | Res: ≧50%, + | Res: ≧50%, ±/+ | Res: ≧50%, ± |
| 1/8 | M: ≧50%, ++ | M: ≧50%, ±/+ | M: ≧50%, −/± |
| | Res: nd | Res: nd | Res: ≧50%, ++ |
| 1/16 | M: nd | M: >50%,++ | M: >50%, ++ |
| | Res: nd | Res: nd | Res: nd |

Solution B: CSSOL1 at pH 3.7
Solution B3: CSSOL1 at pH 3.7 with 150% acetic acid compared to CSSOL1 and Solution B.

TABLE 6
Comparison of spermicidal activities of four pH 4-copper silicate based
solutions (Donor B)

| | | | | LACSSOL-pH 4- |
|---|---|---|---|---|
| | CSSOL1-pH 4 | Solution B3-pH 4 | LACSSOL-pH 4 | 150% |
| Dilution | 1 min | 1 min | 1 min | 1 min |
| 1/1 | M: 0% | M: 0% | M: ≦10%, ± | M: 0% |
| 1/2 | M: 0% | M: 0% | M: ≧80%, ++/+++ | M: 20-30%, ±/+ |
| 1/4 | M: 0% | M: 0% | M: ≧80%, +++ | M: >50%, +++ |
| 1/8 | M: ≧80%, +++ | M: ≧80%, +++ | M: nd | M: nd |
| 1/16 | M: nd | M: nd | M: nd | M: nd |

TABLE 7
Second comparative study of the spermicidal activity of four pH 4-copper
silicate based solutions (Donor C)

| | | | | LACSSOL-pH 4- |
|---|---|---|---|---|
| | CSSOL1-pH 4 | Solution B3-pH 4 | LACSSOL-pH 4 | 150% |
| Dilution | 1 min | 1 min | 1 min | 1 min |
| 1/1 | M: 0% | M: 0% | M: 0% | M: 0% |
| 1/2 | M: 0% | M: 0% | M: ˜50%, ++ | M: ˜15%, ± |
| 1/4 | M: ˜30%, ± | M: 0% | M: +++ | M: +++ |
| 1/8 | M: ˜65%, ++ | M: ˜20%, ±/+ | M: nd | M: nd |
| 1/16 | M: +++ | M: +++ | M: nd | M: nd |

The present invention includes modifications and adaptations apparent to those skilled in the art. Furthermore, throughout the specification, unless the context requires otherwise, the word “comprise” or variations such as “comprises” or “comprising”, will be understood to imply the inclusion of a stated integer or group of integers but not the exclusion of any other integer or group of integers.

1. A method of controlling sperm comprising the step of contacting the sperm with an effective amount of copper silicate.

2. Use of an effective amount of copper silicate as a contraceptive.

3. Use according to claim 2 wherein the copper silicate is applied to a site expected to receive or come into contact with sperm.

4. Use according to claim 3 wherein the site is a body part such as a reproductive organ or part thereof.

5. Use according to claim 3 wherein the site is selected from the group consisting of: part of the reproductive tract, the penis, vagina or cervix.

6. Use according to claim 3 wherein the site is a contraceptive device or agent such as a condom or the like.

7. Use according to claim 6 wherein the site is a sex aid.

8. A method according to claim 1 wherein the effective amount is selected from the group consisting of: 1-10 g, 2-8 g and 4-6 g.

9. A contraceptive formulation comprising copper silicate and a pharmaceutically acceptable carrier.

10. A contraceptive formulation according to claim 9 adapted for topical administration.

11. A contraceptive formulation according to claim 9 or 10 selected from the group consisting of: a liquid, cream, or gel.

12. A contraceptive formulation according to claim 9 wherein the copper silicate is present as a solution.

13. A contraceptive formulation according to claim 9 wherein the copper silicate is present as an acidified solution.

14. A contraceptive formulation according to claim 13 comprising acetic acid.

15. A contraceptive formulation according to claim 13 or 14 with a pH of about 3-6.

16. A contraceptive formulation according to claim 14 wherein the final concentration of acetic acid in the formulation is about 0.1% wt-2% wt.

17. A contraceptive formulation according to claim 9 wherein the copper silicate is present as a micronized solid.

18. A contraceptive formulation according to claim 9 capable of acting as a lubricant.

19. A contraceptive formulation according to any one of claims 9 to 18 wherein the concentration of copper is approximately 0.01%-10% w/w.

20. A contraceptive formulation according to any one of claims 9 to 18 wherein the concentration of copper is approximately 0.05%-0.5% w/w.

21. A contraceptive formulation according to any one of claims 9 to 18 wherein the concentration of copper is approximately 0.05%-0.3% w/w.

22. A contraceptive device comprising an amount of copper silicate that is able to control sperm.

23. A device according to claim 22 wherein the copper silicate is incorporated thereon.

24. A device according to claim 22 wherein the copper silicate is incorporated therein.

25. A device according to any one of claims 22 to 24 in the form of a barrier agent.

26. A device according to claim 25 wherein the barrier agent is selected from the group consisting of: sponges, films, cervical caps, diaphragms and condoms.

27. The use of copper silicate as a spermicide or for the preparation of a formulation for controlling sperm.
